(19) United States
(12) Patent Application Publication
(10) Pub. No.: US 2008/0008997 A1
(43) Pub. Date: Jan. 10, 2008

(54) Proximity ligation assays with peptide conjugate 'burrs' and aptamers for the sensitive detection of spores and cancer cells

(75) Inventors: Matthew Levy, Austin, TX (US); Andrew D. Ellington, Austin, TX (US); Supriya Pai, Austin, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/512,439

(22) Filed: Aug. 30, 2006

Related U.S. Application Data
(60) Provisional application No. 60/712,600, filed on Aug. 30, 2005.

(51) Int. Cl. C12Q 1/68; C07H 21/04

(52) U.S. Cl. 435/6; 536/24.3

(57) ABSTRACT

The present invention includes compositions and methods for the detection of specific targets on a surface that includes one or more peptides and one or more oligonucleotides connected by a joint to a detectable marker, wherein the joint between the peptides, the oligonucleotides or both the peptides and oligonucleotides are immobilized.

This application claims priority to U.S. Provisional Patent Application Ser. No. 60/712,600, filed Aug. 30, 2005, the entire contents of which are incorporated herein by reference. Without limiting the scope of the invention, its background is described in connection with methods of detection

### STATEMENT OF FEDERALLY FUNDED RESEARCH

This invention was made with U.S. Government support under Contract No. UTA05-006 awarded by the Army Research Office. The government has certain rights in this invention.

### TECHNICAL FIELD OF THE INVENTION

The present invention relates in general to the field of methods of detection, and more particularly, to compositions, methods and kits for highly sensitive detection of targets using peptide conjugated particles.

### BACKGROUND OF THE INVENTION

The sensitive and accurate detection of spores is of key importance for both clinical and biodefense applications. Because of their extraordinary sensitivities (1), PCR-based methods are widely used for the detection and identification of nucleic acid sequences associated with spores (2-4). However, the accurate identification of specific bacterial species often requires that multiple gene targets be detected in parallel (3); otherwise, the amplification of genes from closely related, non-target organisms can occur (5). In addition, the detection of protein as well as nucleic acid targets can help to guard against the detection of false positives. Alternate methods for spore detection have relied upon either ELISA (6) or the binding of fluorescently-labeled antibodies or peptides to spore surfaces, followed by microscopy or FACS (7-9).

Early detection of cancer is very important for accelerated cure and remedy. The use of available detection methods such as tumor biopsies, tissue staining etc are time-intensive, invasive and may be prone to errors due to the heterogeneous nature of tumors (Lee & Thorgeirsson, 2005). This makes the use of cancer cell detection based on unique cell surface antigens a very attractive tool. Aptamers are very effective tools that can be applied in the form of small-molecular detection probes, target inhibitors or target binders. Their relatively small nature allows for easy manipulation and complementation of aptamers with other molecules such as quantum dots, oligonucleotides, nanoparticles and siRNA delivery (Farokhzad et al., 2006). As such, aptamers also serve as excellent biomarker sensors due to their highly specific nature (Famulok et al., 2000). Accordingly, we have applied the sensitive nature of aptamers to the detection of tumor cell surface markers coupled with a unique and high-throughput technique called the “Proximity Ligation Assay (PLA).”

### SUMMARY OF THE INVENTION

The present invention includes compositions, kits and methods of detection that are highly sensitive for surface targets using peptide conjugated particles, aptamer-bound DNA probes and the polymerase chain reaction of amplicons. Since most protein detection methods are not as sensitive as PCR, the present inventors coupled methods for the identification of specific markers on surfaces, e.g., the surfaces of spores, cells, cancer cells, tissue, cell fragments, viruses, viral particles, membranes and the like with PCR amplification. While an immuno-PCR approach should be possible (10), such methods require that unbound antibody-DNA conjugates be separated from bound conjugates, and are inherently prone to generating false positive results due to non-specific binding. Therefore, a proximity ligation assay (11, 12) was further adapted to couple spore coat recognition and real-time PCR amplification. Proximity ligation is an innovative technique in which small DNA tags are co-localized on a protein surface and subsequently ligated together, creating a unique amplicon that can be sensitively detected using real-time PCR. PLA has previously been used to detect zeptomole amounts of proteins (11).

The present inventors recognized that the co-localization of DNA tags on a cell surface, rather than on a single protein molecule, might lead to the specific and sensitive detection of cells. In the present study, peptides were adapted that bind specifically to either Bacillus anthracis, Bacillus subtilis or Bacillus cereus spores (9, 13) to PLA. Peptides and DNA tags were conjugated to the fluorescent protein phycoerythrin (PE), creating multivalent ‘burrs’ that could detect spore surfaces. Following ligation, the amplicons associated with burrs could be used to specifically detect as few as 100 B. anthracis and 10 B. subtilis spores, and down to 1 B. cereus spore. In addition to this, aptamer-conjugated PLA probes were also adapted to the detection of the PSMA positive prostate cancer cell line LNCaP. LNCAP cells as few as 10 cells could be detected not only by themselves but also in a mixture of 100,000 non-cognate and PSMA negative HeLa cells.

### BRIEF DESCRIPTION OF THE DRAWINGS

For a more complete understanding of the features and advantages of the present invention, reference is now made to the detailed description of the invention along with the accompanying figures and in which:

FIG. 1 includes micrographs that show the specificity of monovalent and polyvalent probes. Fluorescent probes were constructed using the NH-peptide (BS-specific). BS and BC spores were incubated with either (FIG. 1a) monovalent NH-peptide:fluorescein conjugates or (FIG. 1b) polyvalent NH-peptide:PE conjugates. Specific binding was only observed when the polyvalent NH-peptide:PE probes were used. Spores were visualized using differential interference microscopy (DIC) and fluorescence microscopy with either fluorescein (FITC) or Texas Red filter sets (TR);

FIG. 2 shows the construction and ligation of burrs. (FIG. 2a) Burrs. Oligonucleotides and peptides are separately conjugated to PE. There are two distinct oligonucleotide conjugates, one linked through its 5′ end and one linked through its 3′ end. (FIG. 2b) Burr ligation and amplification. When simultaneously bound to a spore target, burrs can be aligned by a splint oligonucleotide and ligated to generate a unique amplicon.

FIG. 3 is a graph that shows the optimization of PLA probe concentration for the detection of B. cereus spores. The real-time PCR data represents a single data set in which the probe concentration was varied from 0.1 to 100 pM. PLA reactions conducted in the presence 100 BC spores are indicated by a solid line and those conducted in the absence of spores by dashed lines. A positive, spore-dependent signal was only observed when reactions were conducted using 10 pM probe (bolded);

FIG. 4 includes 3 graphs that show the optimization of PLA probe concentration for 100 B. subtilis and B. cereus spores. A splint concentration of 10 pM was used. The cycle difference represents the difference between the C[T] value of the background amplification reaction (no spores) and amplification in the presence of spores. Reactions containing BS spores and BC spores were carried out with burrs that presented either the NH- (BS-specific) or S-peptides (BC-specific). Reactions containing BA (Sterne) spores were carried out with burrs presenting either the NH-, S- or the ATY-peptides (BA-specific);

FIG. 5 includes 3 graphs that show the splint optimization for 100 spores. A probe concentration of 1 pM was used. Reactions contained burrs as described in FIG. 4. Cycle difference is as in FIG. 4.

FIG. 6 includes 3 graphs that show the specificity of spore detection assays. Reactions were carried out with 10 pM probe and 10 pM splint, and contained burrs bearing one of the three spore-specific peptides. Cycle difference is as in FIG. 4.

FIG. 7 is a graph that shows the number of spores and specificity in the limit of detection for a single BC spore.

For a complete understanding of the applications of the aptamer-probe construct, the following figures are illustrated along with a brief description.

FIG. 8 includes the setup of the anti-PSMA aptamer-probe construct. The anti-PSMA aptamers are extended by the addition of a 3′ and a 5′ DNA extension piece which is complementary to the PLA 3′ and 5′ probe respectively. PLA probes are annealed to the extended aptamers and these specifically bind their target. When in proximity to one another on the target surface, the addition of a connector nucleotide ligates the PLA probes together and the resulting amplicon is detected via real-time PCR thus detecting the target that the aptamer-probes bind.

FIG. 9 includes the binding assay data performed using the extended aptamer to test for continued binding to their target. LNCaP cells were incubated with radio-labeled anti-PSMA aptamer with the 3′ and the 5′ extensions and binding affinity was analyzed as a function of bound aptamer to that of the unbound aptamer. Unextended anti-psma aptamer was used as a positive control.

FIG. 10a, 10b and 10c are graphs that shows the optimization of the aptamer-probe concentrations over three different splint concentrations 400 pM, 40 pM and 4 pM for efficient detection of 1000 psma-positive LNCaP cells versus 1000 psma-negative PC3 cells. Signals are depicted as a function of cycle threshold i.e. Delta C(T) calculated by subtracting the C(T) value of samples with target (cells) from samples without cells. For almost all the aptamer-probe concentrations and splint concentrations, cell specific signals are observed in the real-time reaction.

FIG. 11 depicts graphs representing the PLA detection of 1000, 100 and 10 LNCaP cells in a mixture of 10⁵ non-cognate HeLa cells. The assays have been performed over a range of aptamer-probe concentrations ranging from 1 nM to 10 pM for the detection of 1000 and 100 cells and 1 nM to 0.1 pM range for the detection of 10 cells. All the assays were performed with a constant splint concentration of 400 pM. Cycle differences and signals are as depicted. Signals are compared to signals from samples with LNCaP cells and HeLa cells alone.

FIG. 12 depicts the detection of 1000 PC3 cells using the anti-PC3 extended aptamer probes (PC304) over a concentration range of 1nM to 1 pM. Splint concentration is held constant at 400 pM. PC3 specific signals are observed at all aptamer-probe concentrations except the lowest concentration. LNCaP cells are not detected by the PC304 aptamer-probe.

FIG. 13 shows the detection of 10 PC3 cells in a mixture of 10⁵ HeLa cells over an aptamer-probe gradient of 1 nM to 1 pM. The PC304 aptamer-probe is able to detect its target over all four aptamer-probe concentrations while specifically not being able to recognize non-specific HeLa cells.

FIG. 14 shows the specificity of the PC304 aptamer-probe for only the PC3 cell line and can discriminate it from the other two prostate cancer cell lines used i.e. LNCaP and Du145. Over an aptamer-probe gradient of 1 nM to 1 pM, 1000 PC3 cells were detected while samples with LNCaP and Du145 cells showed no cell-specific signals.

FIG. 15 shows the Failure to detect DU145 prostate cancer cells via anti-PC3 aptamer based PLA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “aptamer” refers to an oligonucleotide that has been designed or discovered that is able to specifically bind a target sequence. The term aptazyme is used to describe an aptamer that also contains catalytic activity against nucleic acids or other targets.

As used herein the terms “protein”, “polypeptide” or “peptide” refer to compounds comprising amino acids joined via peptide bonds and are used interchangeably.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library”. A peptide and/or aptamer may be “target-specific” in that it binds or interacts with its targets above detectable noise in a sample. Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected sequence.

The term “labeled” as used herein indicates that there is some method to visualize or detect the bound probe, whether or not the probe directly carries some modified constituent. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities.

As used herein, the terms “markers,” “detectable markers” and “detectable labels” are used interchangeably to refer to compounds and/or elements that can be detected due to their specific functional properties and/or chemical characteristics, the use of which allows the agent to which they are attached to be detected, and/or further quantified if desired, such as, e.g., an enzyme, radioisotope, electron dense particles, magnetic particles or chromophore. There are many types of detectable labels, including fluorescent labels, which are easily handled, inexpensive and nontoxic.

Multiple methods of probe detection may be used with the present invention, e.g., the binding patterns of different components of the probe may be distinguished—for example, by color or differences in wavelength emitted from a labeled probe.

Polymerase Chain Reaction (PCR) and Real-Time PCR. U.S. Pat. Nos. 4,683,202, 4,683,195, 4,800,159, and 4,965,188, relevant portions incorporated herein by reference disclose conventional PCR techniques. PCR typically employs at least one oligonucleotide primer that binds to a selected nucleic acid template (e.g., DNA or RNA). Primers useful in the present invention include oligonucleotide primers capable of acting as a point of initiation of nucleic acid synthesis within or adjacent to oligonucleotide sequences. A primer can be made from a variety of conventional methods, e.g., synthetically. Primers are typically single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular species of microbe such as, e.g., B. anthracis, or can be designed to amplify a sequence from more than one species of microbe. Primers that can be used to amplify a nucleotide sequence from more than one species are referred to herein as “universal primers.”

PCR assays can employ template nucleic acids such as DNA or RNA, e.g., messenger RNA (mRNA). The template nucleic acid of the present invention may be incorporated into one or more burrs, as described herein below. Template DNA or RNA is created as disclosed herein as part of a proximity ligation assay (PLA) using the techniques disclosed herein, including the use of a nucleic acid split to create a longer amplicon. Nucleic acids can be obtained from any of a number of sources, including plasmids, bacteria, yeast, organelles, and higher organisms such as plants and animals. Standard conditions for generating a PCR product are well known in the art.

Examples of detectable markers include, e.g., fluorescein isothiocyanate (FITC), phycoerythrin (PE), allophycocyanin (APC), Texas Red, PE-CY5 or peridinin chlorophyll protein (PerCP) and cyanine. Additional examples include fluorochrome selected from the group consisting of 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

The detectable markers may serve as a scaffold and at the same time be detectable. In other embodiments, the burrs may be formed of a scaffold, e.g., proteins or molecule, e.g., streptavidin, β-galactosidase, Green Fluorescent Protein (GFP) or albumins, e.g., BSA, hemoglobin (or its subunits), keyhole limpet hemocyanin (KLH), Hen egg lysozyme (HEL), etc. In addition, other materials may function as scaffolding for the burrs disclosed herein, e.g., dendrimers (PAMAM and others), micro- or nano-particles such as polystyrene latex (PSL), polylactic acid, or even the polyvalent surface of quantum dots which could be used for this purpose. In certain embodiment, the burr scaffolding will be biocompatible and/or biodegradable.

A number of targets may be detected using the present invention, e.g., bacteria and/or bacterial debris or a fluid infected with the bacteria may be: Bacillaceae, Mycobacteriaceae, Rhodospirillaceae, Chromatiaceae, Chlorobiaceae, Myxococcaceae, Archangiaceae, Cystobacteraceae, Polyangiaceae, Cytophagaceae, Beggiatoaceae, Simonsiellaceae, Leucotrichaceae, Achromatiaceae, Pelonemataceae, Spirochaetaceae, Spirillaceae, Pseudomonadaceae, Azotobacteraceae, Rhizobiceae, Methylomonadaceae, Halobacteriaceae, Enterobacteriaceae, Vibrionaceae, Bacteroidaceae, Neisseriaceae, Veillonellaceae, bacterial organisms oxidizing ammonia or nitrite, bacterial organisms metabolizing sulfur and sulfur compounds, bacterial organisms depositing iron or manganese oxides, Siderocapsaceae, Methanobacteriaceae, Aerobic and facultatively anaerobic Micrococcaceae, Streptococcaceae, Anaerobic Peptococcaceae, Lactobacillaceae, Coryneform group of bacteria, Propionibacteriaceae, Actinomycetaceae, Frankiaceae, Actinoplanaceae, Dermatophilaceae, Nocardiaceae, Streptomycetaceae, Micromonosporaceae, Rickettsiaceae, Bartonellaceae, Francisellaceae, Yersiniaceae, Clostridiaceae, Anaplasmataceae, Chlamydiaceae, Mycoplasmataceae, Acholeplasmataceae and mixtures or combinations thereof.

Alternatively, the target may be a virus and/or a virus-infected cell or fluid with a virus and/or virus infected cell, e.g., Hepatitis A virus, Hepatitis B virus, Hepatitis C virus, Hepatitis D virus, Hepatitis E virus, human immunodeficiency virus, variola major, Enterovirus, Cardiovirus, Rhinovirus, Aphthovirus, Calicivirus, Orbivirus, Reovirus, Rotavirus, Abibirnavirus, Piscibirnavirus, Entomobirnavirus, Rubivirus, Pestivirus, Flavivirus, Influenzavirus, Pneumovirus, Paramyxovirus, Morbillivirus, Vesiculovirus, Lyssavirus, Coronavirus, Bunyavirus, Herpesvirus, Hantavirus, Alphavirus, Filovirus, Arenavirus and mixtures or combinations thereof.

In yet another example, the present invention may be used for the detection, evaluation and typing of eukaryotic cells. For example, the present invention may be used for tissue typing and the identification of cancer and any other techniques in which the identification of cell surface makers is importance. In fact, certain non-destructive methods may be used that include the delivery of the burrs of the present invention by attaching to them one or more cellular toxin subunits that facilitate transfer into the cytoplasm. Other methods include the destruction of the cell membrane upon cell fixation and detection of the remaining cellular scaffolding and/or infrastructure.

The proximity ligation assay (PLA) has previously been used for the sensitive and specific detection of single proteins. In order to adapt PLA methods to the detection of cell surfaces, multivalent peptide:oligonucleotide:phycoerythrin conjugates (‘burrs’) were generated that can bind adjacent to one another on a cell surface and be ligated together to form unique amplicons. Using the present invention and real-time PCR detection of burr ligation events, it was possible to identify specifically as few as 100 Bacillus anthracis, 10 Bacillus subtilis, and 1 Bacillus cereus spore. Burrs should prove to be generally useful for detecting and mapping interactions and distances between cell surface proteins.

Materials and Methods. Bacterial strains and spores. The Bacillus strains used in this study and their sources were as follows: Bacillus subtilis (ATCC 6051) and Bacillus cereus (ATCC 14579) were obtained from the American Type Culture Collection. Spores were produced by growing the respective bacteria (50 μL) in 500 μL of Luria-Bertoni (LB) broth for three days to an optical density at 600 nm (OD₆₀₀) of 1.6-2.0. The culture was then diluted to an OD₆₀₀ of 0.4-0.5 in synthetic replacement sporulation media (SRSM) (14) and incubated at 37° C. on a shaker at 250 rpm for two days. The culture was centrifuged at 10,000×g for 10 min, and the pellet resuspended and lysed in 2 ml of the detergent B-Per (Pierce Biotechnology, Rockford, Ill.) and lysozyme (5 mg/ml). The lysate was placed on a lab rotator for 30 min at room temperature and then sonicated twice using a sonic dismembrator (Fisher Scientific, Hampton, N.H.) with a Branson model 102D horn fitted with a microtip at an amplitude of 15% for two 5 min intervals. The lysate was placed on ice between the two sonications. The sonicated lysate was centrifuged at 18,000×g for 15 min and washed twice with 3 ml of PBS. During the last wash, the pellet was divided into five aliquots. Bacterial spores were separated from cell debris by density gradient centrifugation with sodium diatrizoate (15). Optimal conditions for separation were determined by resuspending the pellets in 2 ml of 25%, 30%, 35%, 40%, or 45% sodium diatrizoate in ddH₂O. The 2 ml solutions were layered over 20 ml of 50% sodium diatrizoate and centrifuged at 11,000 rpm for 45 min at 4° C. The broken vegetative cell debris floating in the supernatant was removed and the spores were washed three times with 2 ml of ddH₂O. While all five concentrations of sodium diatrizoate could be used to separate spores from the broken vegetative cells, optimal separation was observed for pellets that were resuspended in 35% sodium diatrizoate.

Bacillus anthracis Sterne (BA) was purchased in the form of a vaccine from Colorado Serum (Denver, Colo.). The detergent-based spore suspension was centrifuged at 10,000×g for 45 min to pellet the BA spores. The spores were washed three times with 1 ml of 1×PBS and resuspended in 1 ml of ddH₂O. All spore preparations were titered using a hemocytometer (Hausser Scientific, Pa.).

Peptides and primers. The spore-binding peptides used for PLA were synthesized by Biosynthesis Incorporated (Lewisville, Tex.). The sequences of the peptides were, NH (B. subtilis specific)=NHFLPKVGGGC-OH (SEQ ID NO.: 1); A-TY (B. anthracis specific)=ATYPLPIRGGGC-OH (SEQ ID NO.: 2); and S (B. cereus specific)=SLLPGLPGGGC-OH (SEQ ID NO.: 3). Fluorescently-labeled peptides were synthesized by conjugation of the C-terminal cysteine with fluorescein maleimide, followed by reverse phase HPLC purification.

DNA probes, splint oligonucleotide, and primers were purchased from IDT (Coralville, Iowa) and were adapted from sequences in (11). The sequence of the DNA probes used were, 3′oligonucleotide probe: 5′-P-GTCATCATTCGAATCGTACTGCAATCGGGTATT-S-3′ (SEQ ID NO.: 4) and 5′oligonucleotide probe: 5′-S-GTGACTTCGTGGAACTATCTAGCGGTGTACGTGAGTGGGCATGTAGCAAGAG G-3′ (SEQ ID NO.: 5), where ‘5′-P’ indicates a phosphate and ‘S’ a thiol modification. The templating oligonucleotide (splint) sequence was 5′-AAGAATGATGA CCCTCTTGCTAAAA-3′ (SEQ ID NO.: 6). The primers for PCR amplification were 5′-GTGACTTCGTGGAACTATCTAGCG-3′ (SEQ ID NO.: 7) and 5′-AATACCCGATTGCAGTA CGATTC-3′ (SEQ ID NO.: 8). For real-time PCR detection, we used the TaqMan assay and the probe 5′-FAM-TGTACGTGAGTGGGCATGTAGCAAGAGG-BHQ-3′ (SEQ ID NO.: 9), where FAM was 6-carboxyfluorescein and BHQ the Black Hole Quencher-1. All primers and probes were suspended in ddH₂O to a final concentration of 1 mM each.

PLA probe synthesis. R-Phycoerythrin (PE) was obtained from Prozyme (San Leandro, Calif.) and was purified from its ammonium sulfate buffer using a Microcon YM-100 filter (Millipore, Mass.) and resuspended in 250 μL of 1×PBS. The protein was activated using the heterobifunctional crosslinker sulfosuccinimidyl-4-(-N-maleimidomethyl) cyclohexane-1-carboxylate (sulfo-SMCC, Pierce, Ill.) as previously described (16). In short, 0.1 mg of PE was incubated with 0.2 mg of Sulfo-SMCC for 1 hr at room temperature. The activated PE was then desalted using a NAP-5 column and resuspended in 1 ml of 1×PBS. The final concentration of the activated PE was calculated using a Nanodrop ND-1000 (Nanodrop, Wilmington, Del.).

Probe conjugation was mediated through the terminal cysteine residue on the peptide and either 3′ or 5′ terminal thiol modifications on the oligonucleotides. Prior to conjugation, the DNA probes were treated with 10 mM of DTT for 30 min at room temperature, desalted using a NAP-5 column, and resuspended in 1×PBS to the desired concentration. Conjugation was achieved by incubating 56 pmoles of activated PE with a mixture of a spore specific peptide (400 pmoles) and either the 5′ or 3′ DNA probe (400 pmoles) in 20 uL of PBS overnight in the dark at 4° C. The phycoerythrin conjugates were desalted using a Microcon YM-100 filter and resuspended in 100 μL of 1×PBS. Probe concentrations were measured using the Nanodrop ND-1000. The approximate stoichiometry of oligonucleotide:peptide:PE was determined for the ATY-conjugate probes by comparing the absorbance of the conjugates at 260 nm, 280 nm, and 566 nm. The stoichiometry was estimated to be 5:3:1.

Fluorescence microscopy. Spore binding assays were prepared by combining ˜10⁸ spores with 40 nM fluorescein-labeled monovalent peptide:fluorescein or polyvalent peptide:PE conjugates in a 20 μL reaction. Samples were incubated for an hour at room temperature in 1×PBS and then washed three times with 100 μL of 1×PBS, 0.5% Tween 20. After each wash, the spores were centrifuged at 820×g at 4° C. for 5 min. Following the final centrifugation step, the spores were resuspended in 50 μl of PBS and fluorescence was detected using a Nikon Eclipse E800 microscope. Single band length excitation filters for FITC (501/16; 535/30) and Texas Red (568/24; 610/40) (Chroma, Vt.) were used to observe the monovalent peptide:fluorescein- and polyvalent peptide:PE-labeled spores, respectively. Spores incubated either without peptides or with unlabeled peptides served as controls for all microscopy studies.

Real-time PCR amplification and optimization. All real-time PCR amplifications were performed with an MJ DNA Engine Opticon (MJ Research, Massachusetts). The reactions were initially optimized using a full-length DNA template (1 pM) that was analogous to the ligated PLA probes. A series of reactions were prepared using concentration gradients of MgCl₂ (4 mM, 5 mM and 6 mM), dNTPs (50 μM, 100 μM and 200 μM) and TaqMan probe concentrations of 75 nM and 100 nM. The buffer conditions for optimal amplification were 100 mM KCl, 5 mM MgCl₂, 40 mM Tris-HCl (pH 8.3), 0.4 units of T4 DNA ligase, 0.2 mM dNTPs, 500 nM primers (3′ and 5′ each), 75 nM TaqMan probe, 80 μM ATP, 0.5×Smart cycler additive (0.1 mg/ml non-acetylated BSA, 75 mM trehalose and 0.1% Tween-20 in 8.5 mM Tris buffer (pH 8.0)) and 1.5 units of Platinum Taq polymerase (Invitrogen, California). All reactions were conducted in a total volume of 50 ul. Real-time PCR was performed as follows; samples were heated to 50° C. for 5 min and then cycled 50 times at, 92° C. for 1 min, 50° C. for 1 min, 72° C. for 1 min. The fluorescence intensity of the reaction was measured at the end of each cycle.

Proximity ligation assay. PLA reactions minus enzymes were assembled at room temperature in 48.3uL of optimized PCR buffer. Following the addition of Platinum Taq polymerase (0.3uL at 5 units/uL), ligation reactions were initiated by the addition of T4 DNA ligase (0.4uL at 1 units/uL). The reaction mixtures were incubated for an additional 5 minutes and then placed in the thermocycler. Studies in which burrs and spores were preincubated for 1 hr in PBS prior to the addition of enzymes showed no apparent effect on signal or detection.

All reactions were repeated a minimum of 3 times and were conducted with at least 2 independent preparations of PE-conjugated probes. The cycle differences reported in all figures represents the cycle difference (C[T]) between the background amplification reaction (no spores) and amplification in the presence of varying amounts of target spores.

PLA optimizations were carried out with reactions containing 100 (FIGS. 3, 4 and 5) and 10 spores (data not shown). The optimal probe concentration was determined for reactions containing 10 pM splint and probe concentrations of 100 pM, 50 pM, 10 pM, 1 pM, and 0.1 pM. The optimal splint concentration was determined for reactions containing 1 pM probe and splint concentrations of 100 pM, 50 pM, 10 pM, 1 pM, and 0.5 pM. Spore detection assays were conducted using optimized conditions, 10 pM PLA probe, and 10 pM splint. Reactions contained 10,000, 1000, 100, 10, 1, or 0 spores.

Results and Discussion. The present invention is the use of peptide conjugate ‘burrs’ for spore recognition and PLA. Proximity ligation assays have previously been shown to be a sensitive and specific method for protein detection and analysis. The method relies on two independent affinity reagents that bear oligonucleotide tails binding in proximity to one another; the oligonucleotides can then be ligated together, yielding an amplicon that can be detected by PCR or other amplification methods. PLA was initially developed using DNA aptamers that either bound to individual subunits of a dimeric protein or to different epitopes on the same protein (11). The method has since been expanded to include antibody:DNA conjugates (11, 12). PLA was further expanded to the use of peptide-based affinity reagents that can bind specifically not to proteins, but to the surfaces of spores.

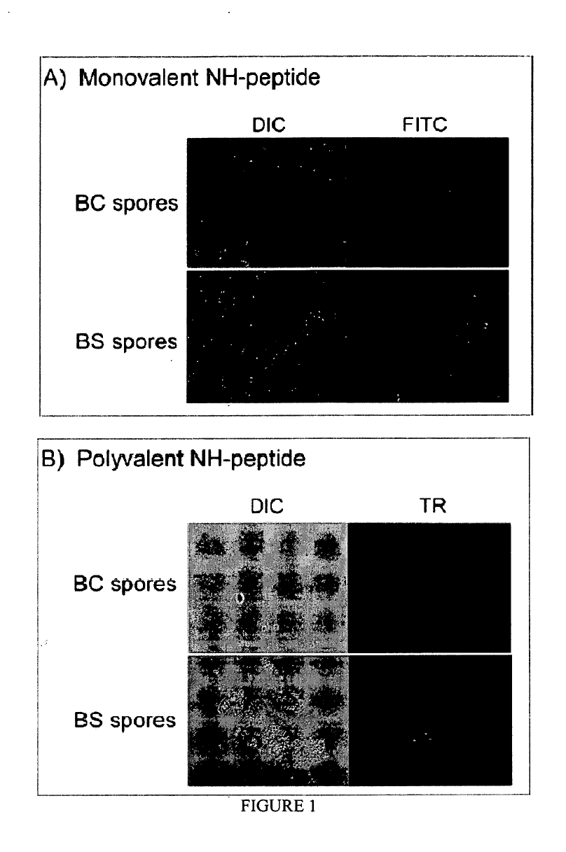

Phage-displayed peptides have been selected that bind with high specificity to several different Bacillus spores (Table I; (9, 13)). It was known that the peptides bound poorly as isolated, synthetic monomers (8, 17), and our own preliminary studies with fluorescent peptide derivatives indicated that there was a significant degree of cross-reactivity between different spores (FIG. 1a). However, polyvalent presentation of the peptides either in the context of a fluorescently-labeled phage or as phycoerythrin (PE) conjugates was known to support specific recognition of spores, and we therefore decided to use phycoerythrin as the basis for PLA affinity reagents. As Turnbough and co-workers previously observed, polyvalent peptide:PE conjugates proved to be highly specific for spores from Bacillus species (FIG. 1b).

TABLE I

Spore-specific peptides used for the design of
PLA probes.

| | | | |
|---|---|---|---|
| Peptide Name | Sequence | Spore-specificity | SEQ ID NO.: |

| | | | |
|---|---|---|---|
| NH | NHFLPKVGGGC-OH | Bacillus subtilis | 1 |
| S | SLLPGLPGGGC-OH | Bacillus cereus, | 3 |
| | | Bacillus thuringiensis | |
| A-TY | ATYPLPIRGGGC | Bacillus anthracis | 2 |

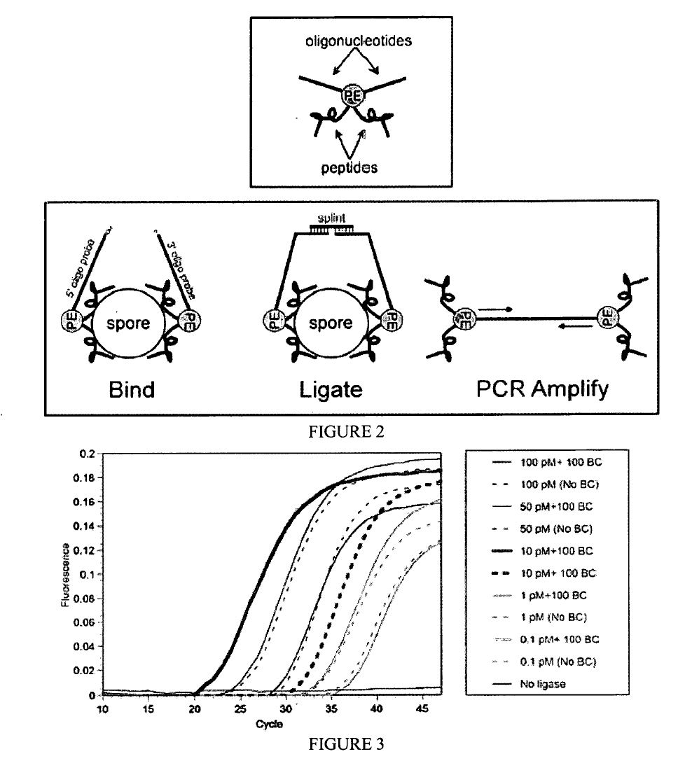

PLA affinity reagents were further developed by conjugating both peptides and oligonucleotides to PE, creating ‘burrs’ that had multiple opportunities to both bind to the spore surface and to position oligonucleotides for ligation reactions (FIG. 2a). Peptides and oligonucleotides bearing thiol linkers were mixed with one another and then with PE activated with sulfo-SMCC. This joint immobilization procedure allows us to control the ratio of peptide:oligonucleotide. Starting with an equimolar ratio of peptide and oligonucleotide resulted in the conjugation of approximately 5 oligonucleotides and 3 peptides per PE. When two burrs bind adjacent to one another on a spore surface, the pendant oligonucleotides can be aligned by an external template (splint) and ligated by T4 DNA ligase. The ligation event can be detected and quantified by real-time PCR (FIG. 2b).

Spore detection via burrs and PLA. Spore-specific burrs were mixed with B. subtilis (BS), B. cereus (BC), or B. anthracis (BA) and incubated for 5 minutes in optimized PLA buffer before the addition of T4 DNA ligase and Taq polymerase. As the intent was to capture preferentially proximity events, ligation was carried out for a very short period of time (5 minutes), and then ligated sequences were amplified via real-time PCR. In addition, since the splint can potentially promote the ligation of the burrs even in the absence of spores we carried out negative controls without spores. Following PCR, the spore-dependent signal is represented as the shift in the number of PCR cycles required for amplification to a given cycle threshold (C[T]) value (18).

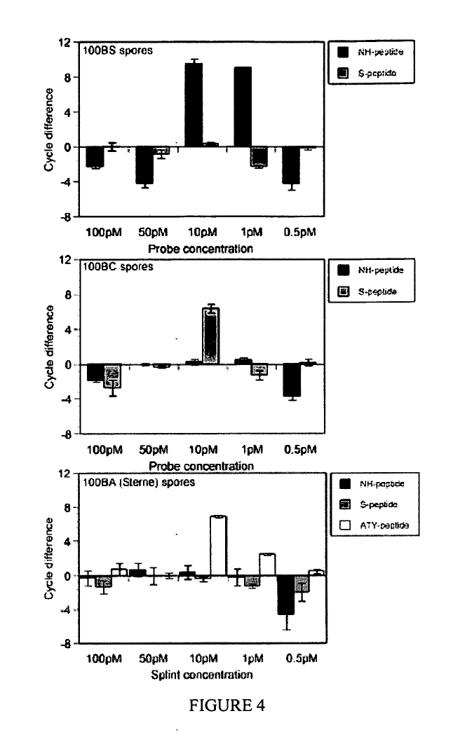

Initially, it was necessary to determine the burr concentration necessary to achieve a significant shift in the cycle threshold. Various burr concentrations from 0.5pM to 100pM were used while keeping the splint concentration (10pM) and other variables constant. PLA reactions with only 100 BS and BC spores were conducted with burrs bearing either the NH- (BS-specific) or S-peptide (BC-specific), while reactions with 100 BA (Sterne) spores were conducted using probes bearing either the NH-, S- or the ATY-peptide (BA-specific). A single data set generated with B. cereus spores is shown in FIG. 3. A substantive real-time PCR signal was observed when the PLA reaction was conducted using a 10 pM concentration of burrs. Similar studies were conducted with spores from all three bacterial species a minimum of 3 times. The averaged data from these studies are shown in FIG. 4. Again, spore-specific signals, indicated by a positive C[T] value, were reproducibly observed at some burr concentrations. PLA reactions in which the burrs and spores were pre-incubated for 1 hr in PBS prior to the addition of enzymes gave similar results (data not shown).

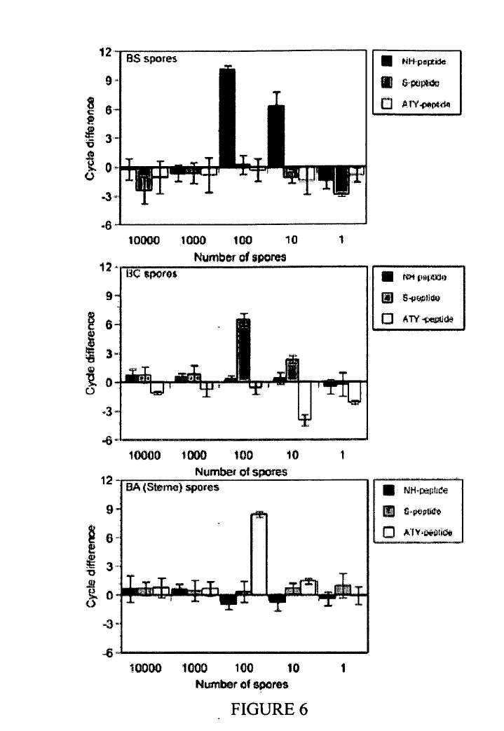

The fact that only some burr concentrations should give large changes in C[T] values is not surprising; too many burrs in solution will yield a background of ligated templates that is not spore-dependent, while too few burrs will not generally bind adjacent to one another on a spore surface, will not ligate, and again will not yield a spore-dependent signal. For 100 spores, 10 pM burr generally seemed to give a reliable signal. Gratifyingly, the BS-specific peptide never yielded a significant, positive C[T] value with BC and BA, and the BC-specific peptide did not give a positive C[T] value with BS or BA. Additional optimizations (FIG. 6) revealed that the BA-specific peptide did not produce a signal in the presence of BS or BC spores. In some cases, a negative cycle difference (˜1-4 cycles) was observed when reactions were conducted in the presence of spores. These negative C[T] differences may reflect the general inhibition of PCR reactions by spores or attendant organics in solution, and further emphasize the validity of the reproducible, positive C[T] values seen with cognate burr:spore pairs.

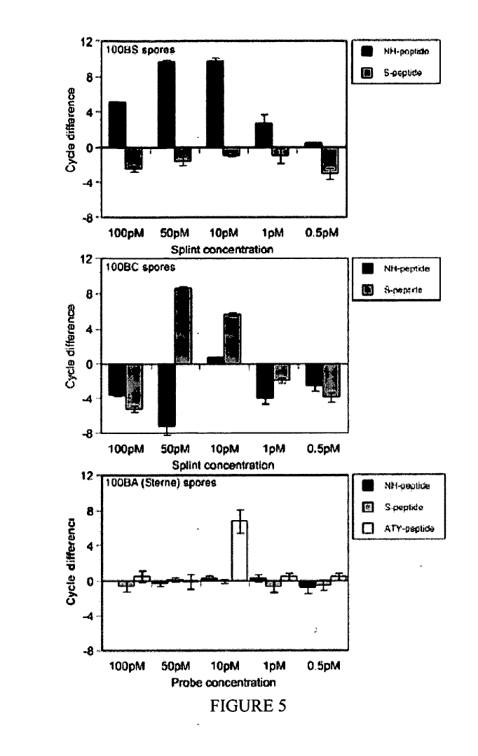

In addition to the affinity reagent concentration, the concentration of the splint oligonucleotide has been shown to be an important factor in the optimization of PLA detection (11, 12). Therefore, we performed a series of assays in which we varied the splint concentration. Assays were conducted using a constant amount (10 pM) of burr and 100 BS, BC or BA (Sterne) spores. As shown in FIG. 5, optimal spore detection was observed for reactions conducted with either 10 pM or 50 pM burr. The decrease in the observed cycle difference at the higher splint concentrations can be attributed to a decrease in the number of amplification cycles necessary to generate a signal in the absence of spores, indicating an increase in the number of spore-independent ligation events (data not shown). Most importantly, though, all reactions conducted with non-cognate spores again showed no positive signal.

Finally, PLA reactions were carried out to examine the limits of detection with burrs. As shown in FIG. 6, specific amplification is once again only observed for each burr with its cognate spore. The observed detection limits for optimized reaction conditions are as few as 10 BC or BS spores, and 100 BA (Sterne) spores. It should again be emphasized that these are detection limits for the detection of the spore coat, not the spore genome, and thus that PLA with burrs is likely the single most sensitive method for the detection of spores themselves currently available.

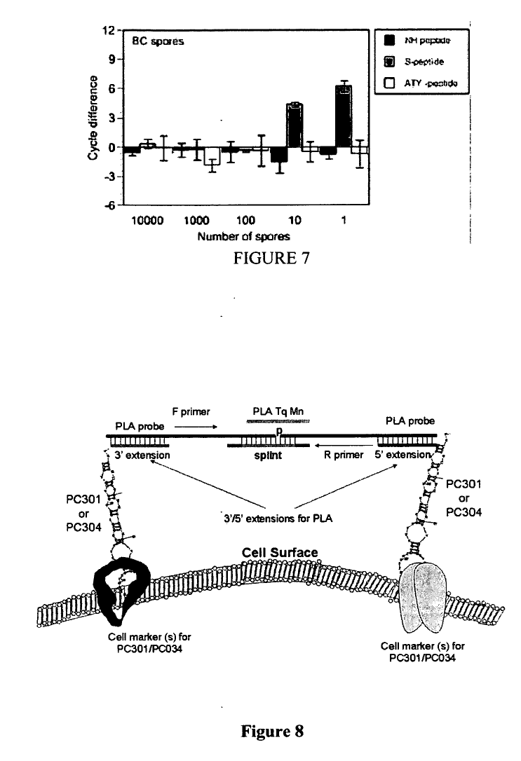

The loss of a positive signal at higher concentrations of spores is likely the due to dilution of the burrs on the spore surface. At higher spore concentrations (10³-10⁴ spores/50 uL the number of burrs binding to adjacent sites on the spore coat is decreased, leading to fewer or no ligation events. In keeping with this hypothesis, we reasoned that at lower spore concentrations there would be fewer spore-dependent ligation events but the same level of background ligation. If so, positive signals would be harder to acquire. Based on this, the PLA detection method was optimized. Splint concentrations were lowered from 10 pM to 1 pM, in order to reduce the level of background ligation. As shown in FIG. 7, the modification resulted in a further decrease in the detection limit to a single BC spore.

FIG. 8 shows the setup of the Anti-PSMA aptamer-probe based PLA. (a) Anti-PSMA aptamers are extended by the addition of a 3′ and a 5′ DNA extension piece which is complementary to the PLA 3′ and 5′ probe respectively. (b) PLA probes are annealed to the extended aptamers and these specifically bind their target. (c) When in proximity to one another on the target surface, the addition of a connector nucleotide ligates the PLA probes together and the resulting amplicon is detected via real-time PCR thus detecting the target that the aptamer-probes bind.

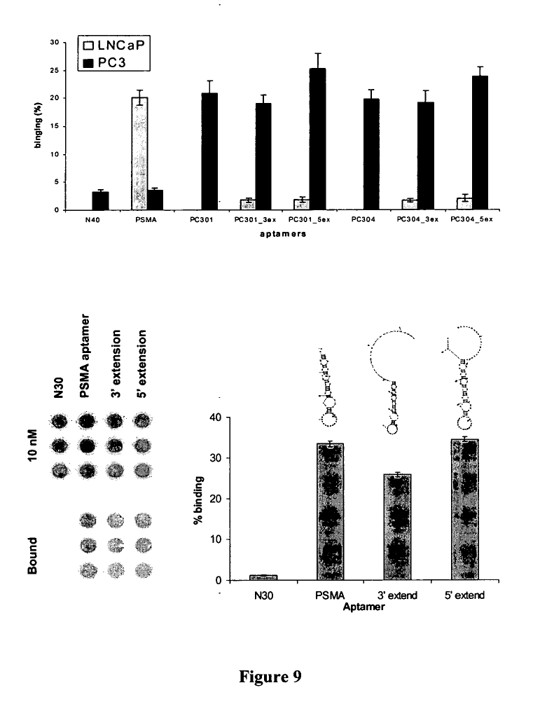

FIG. 9 shows a binding assay data representing the ability of the extended aptamers to continue binding their targets. (A). The anti-PC3 extended aptamers PC301 and PC304 were radiolabeled and incubated with 10⁵ LNCaP and 10⁵ PC3 cells each. The anti-PSMA aptamer was used as a positive control and filter binding assays were performed to test extended aptamer binding. Each sample was assayed in triplicates. (B) The anti-PSMA extended aptamer was radiolabeled and incubated with 10⁵ LNCaP cells to test for aptamer binding to target. Each sample was assayed in triplicates.

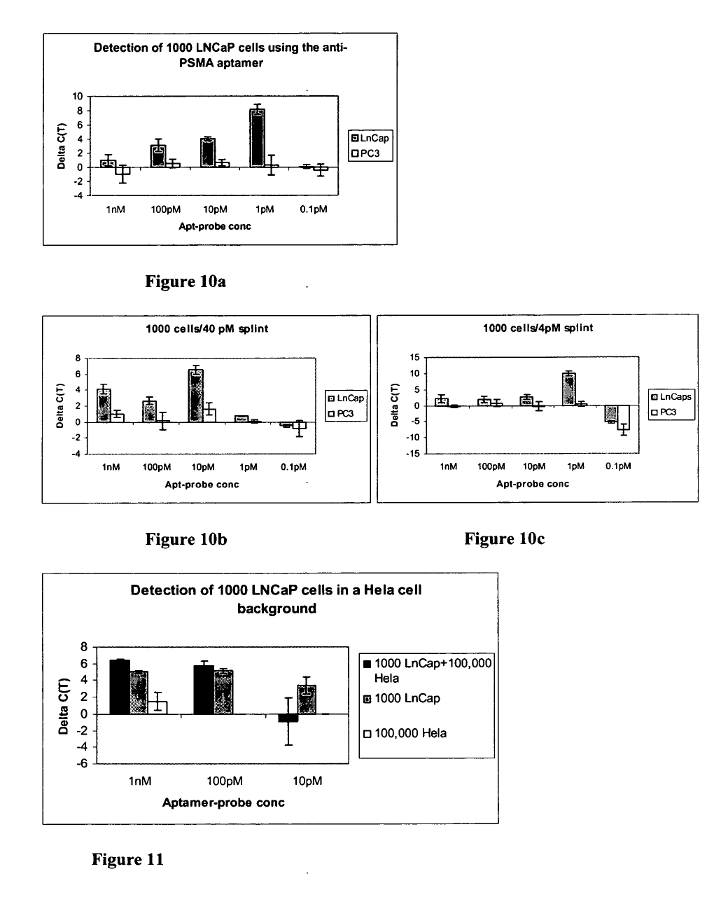

FIG. 10a shows the results from a PLA assay was performed with 1000 LNCaP and 1000 PC3 cells and an aptamer probe concentration gradient ranging from 1 nM to 0.1 pM. Splint concentration was set to 400 pM. The C(T) values of samples containing cells were compared to samples that contained only PBS+. Delta C(T) was calculated by subtracting the C(T) values of samples containing cells from samples containing no cells. Signals were represented in the form of calculated Delta C(T)s.

FIGS. 10b and 10c show the results from PLA assays performed with 1000 LNCaP and 1000 PC3 cells and an aptamer probe concentration gradient ranging from 1 nM to 0.1 pM. Splint concentration was set to 40 pM and 4 pM. The C(T) values of samples containing cells were compared to samples that contained only PBS+. Delta C(T) was calculated by subtracting the C(T) values of samples containing cells from samples containing no cells. Signals were represented in the form of calculated Delta C(T) values.

FIG. 11 is a cell surface PLA was carried out using 1000 LNCaP cells mixed with 10⁵ HeLa cells. Additionally samples containing only 1000 LNCaP cells or only 10⁵ HeLa cells. The extended aptamer probes were incubated with the samples each along with a connector nucleotide (400 pM) and ligated using T4 DNA ligase. The C(T) values of samples containing cells were compared to samples that contained only PBS+. Delta C(T) was calculated by subtracting the C(T) values of samples containing cells from samples containing no cells. Signals were represented in the form of calculated Delta C(T) values.

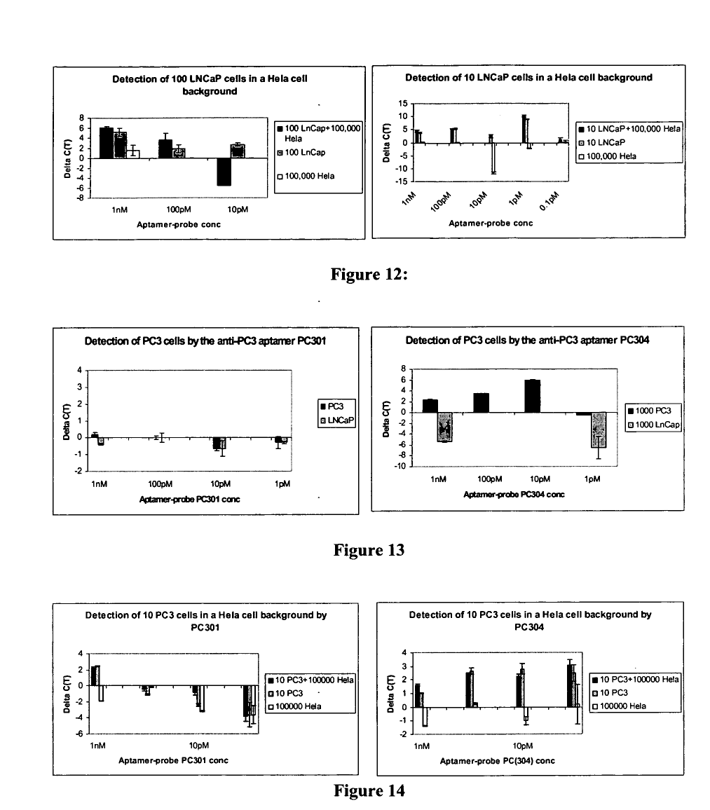

FIG. 12 shows the detection of lower cell number was demonstrated by assaying 100 and 10 LNCaP cells in a HeLa cell background. The extended aptamer probes were incubated with the samples each along with a connector nucleotide (400 pM) and ligated using T4 DNA ligase. The C(T) values of samples containing cells were compared to samples that contained only PBS+. Delta C(T) was calculated by subtracting the C(T) values of samples containing cells from samples containing no cells. Signals were represented in the form of calculated Delta C(T) values.

FIG. 13 shows the detection of 1000 PC3 cells by the anti-PC3 aptamers PC301 and PC304. Cell surface PLA was performed using the two aptamer-probes with 1000 PC3 and LNCaP cells. Delta C(T)s were calculated by subtracting C(T) values of samples with cells from samples without cells

FIG. 14 shows the detection of 10 PC3 cells by PC301 and PC304 in a background of HeLa cells. PLA assays were conducted with 10 PC3 cells combined with 105 HeLa cells. Controls used included 10 PC3 cells and 10⁵ HeLa by themselves. Delta C(T) was calculated by subtracting the C(T) values of samples containing cells from samples containing no cells. Signals were represented in the form of calculated Delta C(T) values.

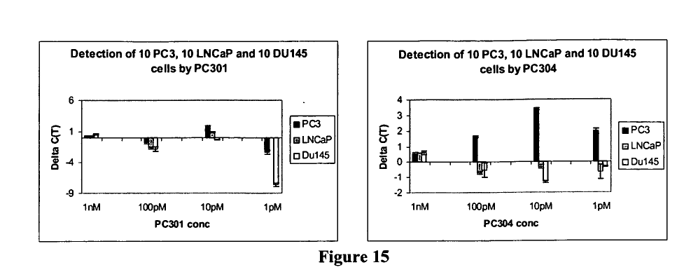

FIG. 15 shows the failure to detect DU145 prostate cancer cells via anti-PC3 aptamer based PLA. PLA assays were conducted using 10 DU145 cells and the PC301 and PC304 at a concentration of 1 nM, 100 pM, 10 pM and 1 pM and a splint concentration of 400 pM. PC3 cells and LNCaP cells were used as controls. Delta C(T) was calculated by subtracting the C(T) values of samples containing cells from samples containing no cells. Signals were represented in the form of calculated Delta C(T) values.

While there appears to be a relatively narrow window in which specific spore-dependent amplification can be achieved, this window can be rationally manipulated and a variety of spore concentrations could potentially be detected by using several different burr:splint pairs in parallel. Each burr:splint pair would form a unique amplicon and would be present at a concentration that had previously been optimized for a given spore concentration. Thus, in a multiplex PCR, each burr:splint pair could detect a particular concentration range of a spore. Additionally, it may prove possible to improve detection by generating burrs that bear two different peptides for the same spore, or by synthesizing burrs with optimal oligonucleotide:peptide ratios.

The use of burrs is not merely an incredibly sensitive assay for cell surface epitopes, but should be an extremely powerful technique to probe the surfaces of cells. While previous implementations of the proximity ligation assay have indicated that multiple epitopes on the same protein or protein oligomer can be detected simultaneously, the technique was extended to multiple epitopes on the surfaces of cells. To the extent that type, number, or distribution of protein or other epitopes that can be identified by affinity reagents is diagnostic for a given cell or cell type, burr-based PLA may provide novel and interesting information about cell biology. For example, proteins that are ensconced within lipid rafts could be readily detected by spores, even if the total concentration of proteins on the cell surface did not change. Similarly, burrs made from Annexin V could be used to identify when phosphotidylserine began to make an appearance on the cell surface, and thus could be used to monitor the earliest stages of apoptosis. As more applications for burr-based PLA are explored, it is even possible that oligonucleotides of differing lengths could be as molecular rulers for probing the distances between target antigens on a cell surface.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Saiki, R. K., Scharf, S., Faloona, F., Mullis, K. B., Horn, G. T., Erlich, H. A. and Arnheim, N. (1985) Enzymatic amplification of beta-globin genomic sequences and restriction site analysis for diagnosis of sickle cell anemia. Science, 230, 1350-1354.

2. Ellerbrok, H., Nattermann, H., Ozel, M., Beutin, L., Appel, B. and Pauli, G. (2002) Rapid and sensitive identification of pathogenic and apathogenic Bacillus anthracis by real-time PCR. FEMS Microbiol Lett, 214, 51-59.

3. Ramisse, V., Patra, G., Garrigue, H., Guesdon, J. L. and Mock, M. (1996) Identification and characterization of Bacillus anthracis by multiplex PCR analysis of sequences on plasmids pXO1 and pXO2 and chromosomal DNA. FEMS Microbiol Lett, 145, 9-16.

4. Ryu, C., Lee, K., Yoo, C., Seong, W. K. and Oh, H. B. (2003) Sensitive and rapid quantitative detection of anthrax spores isolated from soil samples by real-time PCR. Microbiol Immunol, 47, 693-699.

5. Beyer, W., Pocivalsek, S. and Bohm, R. (1999) Polymerase chain reaction-ELISA to detect Bacillus anthracis from soil samples-limitations of present published primers. J Appl Microbiol, 87, 229-236.

6. Blake, M. R. and Weimer, B. C. (1997) Immunomagnetic detection of Bacillus stearothermophilus spores in food and environmental samples. Appl Environ Microbiol, 63, 1643-1646.

7. Zahavy, E., Fisher, M., Bromberg, A. and Olshevsky, U. (2003) Detection of frequency resonance energy transfer pair on double-labeled microsphere and Bacillus anthracis spores by flow cytometry. Appl Environ Microbiol, 69, 2330-2339.

8. Turnbough, C. L., Jr. (2003) Discovery of phage display peptide ligands for species-specific detection of Bacillus spores. J Microbiol Methods, 53, 263-271.

9. Williams, D. D., Benedek, O. and Turnbough, C. L., Jr. (2003) Species-specific ligands for the detection of Bacillus anthracis spores. Appl Environ Microbiol, 69, 6288-6293.

10. Sano, T., Smith, C. L. and Cantor, C. R. (1992) Immuno-PCR: very sensitive antigen detection by means of specific antibody-DNA conjugates. Science, 258, 120-122.

11. Fredriksson, S., Gullberg, M., Jarvius, J., Olsson, C., Pietras, K., Gustafsdottir, S. M., Ostman, A. and Landegren, U. (2002) Protein detection using proximity-dependent DNA ligation assays. Nat Biotechnol, 20, 473-477.

12. Gullberg, M., Gustafsdottir, S. M., Schallmeiner, E., Jarvius, J., Bjarnegard, M., Betsholtz, C., Landegren, U. and Fredriksson, S. (2004) Cytokine detection by antibody-based proximity ligation. Proc Natl Acad Sci U S A, 101, 8420-8424.

13. Knurr, J., Benedek, O., Heslop, J., Vinson, R. B., Boydston, J. A., McAndrew, J., Kearney, J. F. and Turnbough, C. L., Jr. (2003) Peptide ligands that bind selectively to spores of Bacillus subtilis and closely related species. Appl Environ Microbiol, 69, 6841-6847.

14. Jinks, D. C., Guthrie, R. and Naylor, E. W. (1985) Simplified procedure for producing Bacillus subtilis spores for the Guthrie phenylketonuria and other microbiological screening tests. J Clin Microbiol, 21, 826-829.

15. Nicholson, W. L. and Setlow, P. (1990), Molecular microbiological method for Bacillus. John Wiley & Sons, Ltd, Chichester, United Kingdom, pp. 391-450.

16. Hermanson, G. T. (1995) Bioconjugate techniques. Acedemic Press.

17. Turnbough, C. L., Jr. (2004).

18. Martell, M., Gomez, J., Esteban, J. I., Sauleda, S., Quer, J., Cabot, B., Esteban, R. and Guardia, J. (1999) High-throughput real-time reverse transcription-PCR quantitation of hepatitis C virus RNA. J Clin Microbiol, 37, 327-332.

19. Famulok M, Mayer G, Blind M. 2000. Nucleic acid aptamers—from selection in vitro to applications in vivo. Acc Chem Res 33:591-599.

20. Farokhzad O C, Karp J M, Langer R. 2006. Nanoparticle-aptamer bioconjugates for cancer targeting. Expert Opin Drug Deliv 3:311-324.

21. Lee J S, Thorgeirsson S S. 2005. Genetic profiling of human hepatocellular carcinoma. Semin Liver Dis 25:125-132.

What is claimed is:

1. A detectable marker comprising:
one or more peptides and one or more oligonucleotides connected by a chemical bond to a detectable marker, wherein the chemical bond between the peptides, the oligonucleotides or both the peptides and oligonucleotides are immobilized and either the peptide or the oligonucleotides or both are target-specific.

2. The marker of claim 1, wherein a ratio between peptides and oligonucleotides is 1:10, 3:5, 1:1, 5:3 or 10:1.

3. The marker of claim 1, wherein a ratio between peptides and oligonucleotides is about equimolar.

4. The marker of claim 1, wherein the detectable marker is fluorescein isothiocyanate (FITC), phycoerythrin (PE), allophycocyanin (APC), Texas Red, PE-CY5 or peridinin chlorophyll protein (PerCP) and cyanine.

5. The marker of claim 1, wherein the target comprises a bacteria selected from the group consisting of Bacillaceae, Mycobacteriaceae, Rhodospirillaceae, Chromatiaceae, Chlorobiaceae, Myxococcaceae, Archangiaceae, Cystobacteraceae, Polyangiaceae, Cytophagaceae, Beggiatoaceae, Simonsiellaceae, Leucotrichaceae, Achromatiaceae, Pelonemataceae, Spirochaetaceae, Spirillaceae, Pseudomonadaceae, Azotobacteraceae, Rhizobiceae, Methylomonadaceae, Halobacteriaceae, Enterobacteriaceae, Vibrionaceae, Bacteroidaceae, Neisseriaceae, Veillonellaceae, bacterial organisms oxidizing ammonia or nitrite, bacterial organisms metabolizing sulfur and sulfur compounds, bacterial organisms depositing iron or manganese oxides, Siderocapsaceae, Methanobacteriaceae, Aerobic and facultatively anaerobic Micrococcaceae, Streptococcaceae, Anaerobic Peptococcaceae, Lactobacillaceae, Coryneform group of bacteria, Propionibacteriaceae, Actinomycetaceae, Frankiaceae, Actinoplanaceae, Dermatophilaceae, Nocardiaceae, Streptomycetaceae, Micromonosporaceae, Rickettsiaceae, Bartonellaceae, Francisellaceae, Yersiniaceae, Clostridiaceae, Anaplasmataceae, Chlamydiaceae, Mycoplasmataceae, Acholeplasmataceae and mixtures or combinations thereof.

6. The marker of claim 1, wherein the target comprises a virus selected from the group consisting of Hepatitis A virus, Hepatitis B virus, Hepatitis C virus, Hepatitis D virus, Hepatitis E virus, human immunodeficiency virus, variola major, Enterovirus, Cardiovirus, Rhinovirus, Aphthovirus, Calicivirus, Orbivirus, Reovirus, Rotavirus, Abibirnavirus, Piscibirnavirus, Entomobirnavirus, Rubivirus, Pestivirus, Flavivirus, Influenzavirus, Pneumovirus, Paramyxovirus, Morbillivirus, Vesiculovirus, Lyssavirus, Coronavirus, Bunyavirus, Herpesvirus, Hantavirus, Alphavirus, Filovirus, Arenavirus and mixtures or combinations thereof.

7. The marker of claim 1, wherein the target is a eukaryotic cell.

8. The marker of claim 1, wherein the target is a cell infected with a pathogen.

9. The marker of claim 1, wherein the target is a cancer cell.

10. The marker of claim 1, wherein oligonucleotides is an aptamer linked to a PLA probe specific to the detection of the PSMA positive prostate cancer cell line LNCaP.

11. The marker of claim 1, wherein the oligonucleotide comprises an aptamer.

12. A method of detection comprising the steps of:
contacting target-specific burrs with a potential target;
adding a DNA ligase and a DNA polymerase in the presence of nucleotides;
optionally adding a nucleic acid splint; and
performing an extension reaction.

13. The method of claim 12, wherein the burr comprises one or more peptides and one or more oligonucleotides connected by a joint to a detectable marker, wherein the joint between one or both the peptides and oligonucleotides is immobilized.

14. The method of claim 12, wherein the ligase is a T4 DNA ligase.

15. The method of claim 12, wherein the DNA polymerase is a Taq polymerase.

16. The method of claim 12, wherein the target is a bacterial cell, a eukaryotic cell, a spore or a virus.

17. The method of claim 12, wherein the detectable marker is fluorescein isothiocyanate (FITC), phycoerythrin (PE), allophycocyanin (APC), Texas Red, PE-CY5 or peridinin chlorophyll protein (PerCP) and cyanine.

18. The method of claim 12, wherein the target number in a mixture is 100 or less.

19. The method of claim 12, wherein the detectable marker is a fluorochrome selected from the group consisting of 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

20. A method for detecting the presence, absence, or amount of one or more targets used in bioterrorism comprising the steps of:
providing a sample obtained from an environment susceptible to bioterrorism attack or an environment within which a bioterrorism attack has taken place; and
detecting the presence, absence, or amount of the target by:
contacting target-specific burrs with a potential target;
adding a DNA ligase and a DNA polymerase in the presence of nucleotides;
optionally adding a nucleic acid splint; and performing an extension reaction.

21. The method of claim 20, wherein the burr comprises one or more peptides and one or more oligonucleotides connected by a joint to a detectable marker selected from fluorescein isothiocyanate (FITC), phycoerythrin (PE), allophycocyanin (APC), Texas Red, PE-CY5 or peridinin chlorophyll protein (PerCP) and cyanine, wherein the joint between the peptides, the oligonucleotides or both the peptides and oligonucleotides is immobilized.

22. A kit at least one vial comprising:
a target-specific burr comprising one or more peptides and one or more oligonucleotides connected by a joint to a detectable marker, wherein the joint between the peptides, the oligonucleotides or both the peptides and oligonucleotides are immobilized and are specific for the target.

23. A detectable marker comprising:
one or more peptides and one or more oligonucleotides connected by a joint to scaffold, one or more a detectable markers attached to the scaffold, wherein the joint between the peptides, the oligonucleotides or both the peptides and oligonucleotides are immobilized.

24. A proximity ligation assay oligo-receptor conjugate for cell surface analysis.


SEQUENCE LISTING (9 sequences)

SEQ ID NO: 1 - protein, 11 aa, Bacillus subtilis
NHFLPKVGGGC

SEQ ID NO: 2 - protein, 12 aa, Bacillus anthracis
ATYPLPIRGGGC

SEQ ID NO: 3 - protein, 11 aa, Bacillus cereus
SLLPGLPGGGC

SEQ ID NO: 4 - DNA, 33 bp, Artificial
  artificail sequence
  misc_structure (1)..(1): 5 prime phosphate
  misc_structure (33)..(33): 3 prime thiol
gtcatcattcgaatcgtactgcaatcgggtatt

SEQ ID NO: 5 - DNA, 53 bp, Artificial
  Artificial Sequence
  misc_structure (1)..(1): 5 prime thiol
gtgacttcgtggaactatctagcggtgtacgtgagtgggcatgtagcaagagg

SEQ ID NO: 6 - DNA, 25 bp, Artificial
  Artifical Sequence
aagaatgatgaccctcttgctaaaa

SEQ ID NO: 7 - DNA, 24 bp, Artificial
  Artificial Sequence
gtgacttcgtggaactatctagcg

SEQ ID NO: 8 - DNA, 23 bp, Artificial
  Artificial Sequence
aatacccgattgcagtacgattc

SEQ ID NO: 9 - DNA, 28 bp, Artificial
  Artificial Sequence
  misc_structure (1)..(1): 5 prime 6-carboxyfluorescein (FAM)
  misc_structure (28)..(28): 3 prime Black Hole Quencher 1 (BHQ1)
tgtacgtgagtgggcatgtagcaagagg